(19) United States
(12) Patent Application Publication
(10) Pub. No.: US 2016/0199146 A1
(43) Pub. Date: Jul. 14, 2016

(54) STEREOTACTIC STABILIZER

(71) Applicant: REFERENCE TECHNOLOGY LIMITED COMPANY, Taichung City (TW)

(72) Inventors: Chun-Hwei TAI, New Taipei City (TW); Ya-Yu TSOU, Taichung City (TW); Ching-Te CHEN, New Taipei City (TW); Sheng-Hong TSENG, Taipei City (TW)

(21) Appl. No.: 14/993,749

(22) Filed: Jan. 12, 2016

(30) Foreign Application Priority Data
Jan. 14, 2015 (TW) 104200569

(51) Int. Cl. A61B 90/10

(52) U.S. Cl. CPC A61B 90/10; A61B 2090/101

(57) ABSTRACT

A stereotactic stabilizer is adapted to be mounted to a fixing device and to be mounted with a stereotactic surgical instrument. The stereotactic stabilizer includes base seat and a rod member. The base seat has a main portion and an instrument connecting portion that is provided on the main portion and that is adapted to be mounted with the stereotactic surgical instrument. The rod member extends from the base seat and is adapted to be mounted to the fixing device so as to be positioned relative to a head of a patient.

### CROSS-REFERENCE TO RELATED APPLICATION

This application claims priority of Taiwanese Application No 104200569 filed on Jan. 14 2015.

### FIELD

The disclosure relates to an auxiliary surgical instrument, and more particularly to a stabilizer adapted for use in a frameless stereotactic surgery.

### BACKGROUND

A stereotactic brain surgery is a surgery that typically relies on CT (computed tomography) or MRI (magnetic resonance imaging) scans for positioning and guiding a fine probe or other similar surgical instruments deep into the brain to conduct biopsy or aspiration procedures at a lesion. The stereotactic brain surgery can reduce damage to critical areas of a patient's brain, prevent the patient from having to bear the risks associated with a craniotomy, and shorten the period of time the patient need spend in ICU (intensive care unit)after the surgery.

As the stereotactic brain surgery requires exceptional precision, the conventional method uses a frame that is fixed onto the patient's skull and that has the surgical instrument mounted thereon to conduct the surgery. However, since the mounting process of the frame onto the patient's skull is time-consuming and generally conducted in the morning to avoid interfering with the operation, patients are essentially forced to wear the heavy frame for the entire day without being permitted to move or turn their heads. To counter the problem of discomfort associated with the conventional method, a frame less stereotactic surgery was introduced, which fixes the surgical instrument directly on the patient's skull and eliminates the necessity of the frame.

The frameless stereotactic surgery can foe exemplified by U.S. Pat. No. 7,794,469B2, which uses a tripod-like surgical instrument having three legs that are fixed onto the skull by way of three nails respectively disposed on the three legs and driven into the skull to achieve the purposes of stabilization and precision. However, the foregoing method does not allow for the surgical instrument to freely move about once the fixation of the surgical instrument has been completed. To treat other lesions located at different areas of the patient's head, the surgical instrument need be removed and relocated, resulting in a relatively large number of holes being left in the skull after the surgery. This not only hinders the post-surgical recovery process of the patient, but also raises the risk of the operation itself.

Furthermore, when applied on the more fragile skulls of children, the foregoing method of stabilizing the surgical instrument upon a head tends to cause brain damage if the nails are driven through the skull to contact the brain. Even if nails of smaller sizes are employed to combat this problem, another problem of insufficient stabilization of the surgical instrument would arise. This issue is prevalent not only with children, but with patients having thin skulls or weak bone strength (e.g. patients with osteoporosis) as well. This issue, combined with the aforementioned relocation issue, seems to indicate that there is still room in the art for improvement.

### SUMMARY

Therefore, an object of the disclosure is to provide a stereotactic stabilizer that can alleviate at least one of the drawbacks of the prior art.

A stereotactic stabilizer is adapted to be mounted to a fixing device and to be mounted with a stereotactic surgical instrument. The stereotactic stabilizer includes a base seat and a rod member. The base seat has a main portion and an instrument connecting portion that is provided on the main portion and that is adapted to be mounted with the stereotactic surgical instrument. The rod member extends from the base seat and is adapted to be mounted to the fixing device so as to be positioned relative to a head of a patient.

### BRIEF DESCRIPTION OF THE DRAWINGS

Other features and advantages of the disclosure will become apparent in the following detailed description of the embodiments with reference to the accompanying drawings, of which:

FIG. 1 is a fragmentary partly exploded perspective view of a first embodiment of the stereotactic stabilizer according to the disclosure, a fixing device and a stereotactic surgical instrument;

FIG. 2 is a perspective view of the first embodiment; and

FIG. 3 is a partly exploded perspective view of a second embodiment of the stereotactic stabilizer according to the disclosure.

### DETAILED DESCRIPTION

Before the disclosure is described in greater detail, it should be noted that like elements are denoted by the same reference numerals throughout the disclosure.

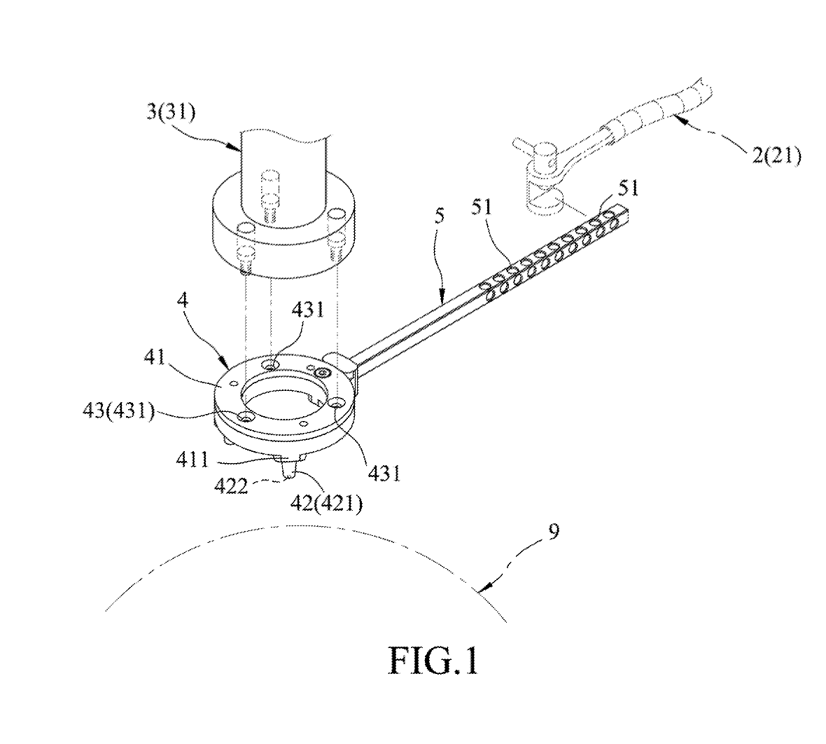

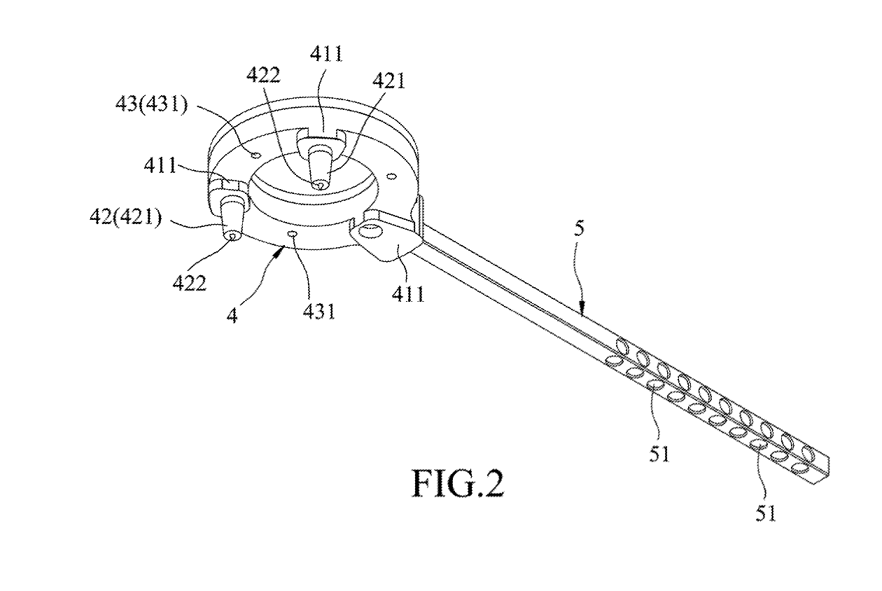

Referring to FIGS. 1 and 2, the first embodiment of the stereotactic stabilizer according to the disclosure is suitable for functioning as an auxiliary surgical instrument for a frameless stereotactic surgery. The stereotactic stabilizer is adapted to be mounted to a fixing device 2 and to be mounted with a stereotactic surgical instrument 3. The fixing device 2 may be a serpentine mounted neuro flexible arm. The stereotactic surgical instrument 3 may include navigational components such as microdrives, probes, etc., endoscopes, electrodes, cables, and so on. The stereotactic surgical instrument 3 is configured as a microdrive throughout the disclosure. Since mounted neuro flexible arms and microdrives are well known in the art, the specifics thereof will not be detailed herein for the sake of brevity.

The stereotactic stabilizer includes a base seat 4 and a rod member 5.

The base seat 4 has a main portion 41, a positioning unit 42, and an instrument-connecting portion 43.

The main portion 41 is ring-shaped and has three protrusions 411 extending from a side surface thereof. An inner diameter of the main portion 41 is configured to be adjustable to accommodate the needs of different operations, and ranges between 2.4 centimeters to 10 centimeters. When the inner diameter is smaller than 2.4 centimeters, not only may there not be enough space for placement of the stereotactic surgical instrument 3, but may also not provide enough vision for an operator during the operation.

The positioning unit 42 is connected to the side surface of the main portion 41 and is adapted to abut against and be positioned relative to a head 9 of a patient. In this embodiment, the positioning unit 42 has two spaced-apart positioning studs 421 that are removably connected to the side surface of the main portion 41, each of the positioning studs 421 having a tapered end section 422 that is distal from the side surface of the main portion 41 and that is adapted to be positioned onto the head 9 of the patient. The positioning studs 421 are respectively and threadedly connected to two of the protrusions 411 via two screws (not shown). It should be noted that the number of the positioning studs 421 may be varied depending on the surgical location, and is not limited to that disclosed herein. Should the operation be performed at an uneven area of the head, only two of the positioning studs 421 may be required; should the operation be performed at a smoother area of the head, three of the positioning studs 421 may be required.

Since the positioning unit 42 is a removable component, the: cleaning process thereof simply involves removing the positioning unit 42 from the main portion 41 and washing the same. Should it be determined that the positioning unit 42 is not needed for the operation at hand, the operator may also choose to remove the positioning unit 42 and conduct the operation therewithout, relying only on the fixing device 2 for stabilization.

The instrument-connecting portion 43 is provided on the main portion 41, is adapted to be mounted with the stereotactic surgical, instrument 3, and has a plurality of spaced-apart threaded holes 431 that are formed in a side surface of the main portion 41 and through an opposite side surface of the main portion 41 (i.e., the threaded holes 431 extend through the main portion 41), and that are adapted to allow threaded connection between the base seat 4 and the stereotactic surgical instrument 3.

The rod member 5 is formed as an elongated rectangular prism, extends from the base seat 4, and is adapted to be mounted to the fixing device 2 so as to be positioned relative to the head 9 of the patient. The rod member 5 is formed with a plurality of indentations 51 that are adapted for facilitating connection between the rod member 5 and the fixing device 2. It should be noted that the rod member 5 may also be cylindrical or in other prismatic shapes, with the indentations 51 being absent or varied in number depending on the method of connection between the rod member 5 and the fixing device 2, and is not limited to that disclosed herein.

To lower the risk of infection due to unsanitary surgical instruments, the stereotactic stabilizer according to the disclosure may be made of plastic material and be disposed after each use. To save medical resources, however, the stereotactic stabilizer may also be made of alloys, such as titanium or aluminum alloys for lighter weight, to facilitate repeated use thereof.

During surgical operation on the patient, the head 9 of the patient is first fixed in place by, e.g., a Mayfieid® skull clamp (not shown), and the rod member 5 is mounted to the fixing device 2. Then, once the base seat 4 has been positioned to the target area on which the operation is to be conducted, the tapered end sections 422 of the positioning unit 42 are abutted against the head 3 of the patient and the fixing device 2 is firmly secured, thereby stabilizing the position of the base seat 4 relative to the head 9. Once the foregoing has been accomplished, the stereotactic surgical instrument 3 can be connected to the main portion 41 of the base seat 4 for commencement of the operation. The stereotactic surgical instrument 3 of the embodiment is connected to the base seat 4 via threaded engagement between three screws and the threaded holes 431, respectively. When there are multiple lesions on the head 9 in need of operation, the operator need only release hold of the fixing device 2 and shift the base seat 4 to another position (i.e. where another lesion is located), then proceed to stabilize the base seat 4 in the same manner described above.

In sum, the stereotactic stabilizer according to the disclosure has the following advantages over conventional equivalents:

1. The stereotactic stabilizer can position/reposition itself anywhere on the head 9 of the patient by virtue of its reliance on only the fixing device 2 and the tapered end sections 422 of the base seat 4 for stabilization, and can be mounted with the stereotactic surgical instrument 3 to thereby fix the stereotactic surgical instrument 3 in place and lower the risk of brain damage to patients of younger age and thinner skulls.

2. The tapered end sections 422 of the positioning unit 42 abut against but do not damage the patient's skull, firmly fixing the base seat 4 in position upon the head 9 during operation, with further stabilization provided by the fixing device 2. This makes the stereotactic stabilizer especially suitable when performing head surgery on children.

3. Since the positioning unit 42 is removable from the base seat 4, it can be separately cleaned to prevent grime from accumulating in the seams and infecting the patient.

4. The operator can choose whether or not to employ the positioning unit 42; should the operator deem them unnecessary for a particular operation, the positioning unit 42 can be removed to rely solely on the fixing device 2 for stabilization thereby providing more flexibility in usage of the stereotactic stabilizer.

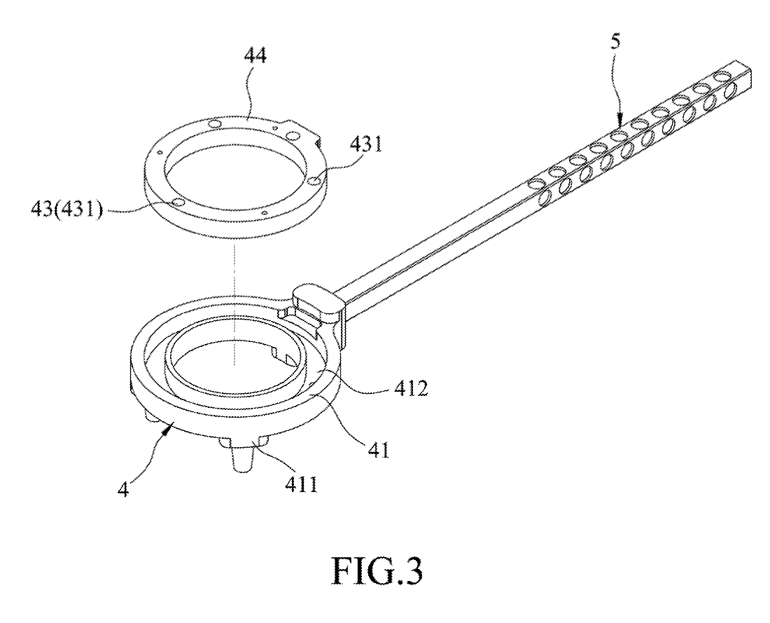

Referring to FIG. 3, the second embodiment of the stereotactic stabilizer according to the disclosure is similar to the first embodiment, except that in the second embodiment, the main portion 41 of the base seat 4 is formed with an annular groove 412, and the base seat 4 further has a replaceable member 44 that is removably connected to the main portion 41 and that is provided with the instrument-connecting portion 43. To be more specific, the replaceable member 44 is ring-shaped and is retained fittingly and removably within the annular groove 412.

The stereotactic surgical instrument 3 can be threadedly connected to the replaceable member 44 via a plurality of screws (see those in FIG. 1), which also fix the replaceable member 44 within the annular groove 412 of the main portion 41.

The replaceable member 44 can be made of a material having lower mechanical strength and cost, such as plastic, aluminum, or aluminum alloy. Given that the primary purpose of the replaceable member 44 is for the stereotactic surgical instrument 3 to be mounted thereon, and since it is made of a lower costing material, the replaceable member 44 may serve as a disposable component that may be replaced after each use. In this way, other components of the embodiment can be made of higher costing materials such as steel or titanium alloys to allow repeated use thereof and to save medical resources.

Therefore, the second embodiment of the stereotactic stabilizer has not only the advantages of the first embodiment, but has also the additional advantages of the replaceable member 44, which is lower in cost and replaceable, so that when the threaded holes 431 of the instrument-connecting portion 43 sustains damage from repeated screwing and unscrewing of the stereotactic surgical instrument 3 thereonto, the replaceable member 44 may simply be replaced with another of its like. This ensures that the stability of the stereotactic surgical instrument 3 is not compromised.

In conclusion, the stereotactic stabilizer according to the disclosure can be stably positioned at any location on the head 9 of the patient and can be mounted with a stereotactic surgical instrument 3 so that no nails need be driven into the skull to achieve stabilization, thereby allowing convenient relocation of the surgical area and minimizing damage to the head 9.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A stereotactic stabilizer adapted to be mounted to a fixing device and to be mounted with a stereotactic surgical instrument, said stereotactic stabilizer comprising:
a base seat having a main portion, and an instrument-connecting portion that is provided on said main portion and that is adapted to be mounted with the stereotactic surgical instrument; and
a rod member extending from said base seat and adapted to be mounted to the fixing device so as to be positioned relative to a head of a patient.

2. The stereotactic stabilizer as claimed in claim 1, wherein said main portion of said base seat is ring-shaped.

3. The stereotactic stabilizer as claimed in claim 2, wherein an inner diameter of said main portion of said base seat ranges between 2.4 centimeters and 10 centimeters.

4. The stereotactic stabilizer as claimed in claim 1, wherein said instrument-connecting portion of said base seat has a plurality of spaced-apart threaded holes that are formed in a side surface of said main portion.

5. The stereotactic stabilizer as claimed in claim 1, wherein said rod member is formed as an elongated rectangular prism, and is formed with a plurality of indentations that are adapted for facilitating connection between said rod member and the fixing device.

6. The stereotactic stabilizer as claimed in claim 1, wherein said base seat further has a positioning unit that is connected to a side surface of said main portion and that is adapted to abut against and be positioned relative to the head of the patient.

7. The stereotactic stabilizer as claimed in claim 6, wherein, said positioning unit includes at least two spaced-apart positioning studs that are removably connected to said side surface of said main portion, each of said positioning studs having a tapered end section that is distal from said side surface of said main portion and that is adapted to be positioned onto the head of the patient.

8. The stereotactic stabilizer as claimed in claim 7, wherein said positioning unit includes three of said positioning studs.

9. The stereotactic stabilizer as claimed in claim 1, wherein said base seat further has a replaceable member that is removably connected to said main portion and that is provided with said instrument-connecting portion.

10. The stereotactic stabilizer as claimed in claim 9, wherein said main portion of said base seat is ring-shaped and is formed with an annular groove, said replaceable member being retained fittingly and removably within said annular groove.
